(19) United States
(12) Patent Application Publication
(10) Pub. No.: US 2014/0249118 A1
(43) Pub. Date: Sep. 4, 2014

(54) TREATMENT OF ORAL LICHEN PLANUS WITH A COMBINATION OF TRIAMCINOLONE AND RETINOIC ACID

(71) Applicants: Shamsoulmolouk Najafi, Tehran (IR); Mahnaz Sahebjamee, Tehran (IR); Massoud Amanlou, Tehran (IR)

(72) Inventors: Shamsoulmolouk Najafi, Tehran (IR); Mahnaz Sahebjamee, Tehran (IR); Massoud Amanlou, Tehran (IR)

(21) Appl. No.: 13/783,326

(22) Filed: Mar. 3, 2013

(51) Int. Cl. A61K 31/573; A61K 31/203

(52) U.S. Cl. CPC A61K 31/573; A61K 31/203
USPC 514/171

(57) ABSTRACT

Oral lichen planus (OLP) is an immunologic disease which can be controlled and cleared by topical application of a composition of triamcinolone acetonide 0.1% and retinoic acid 0.05% in Orabase® on the affected areas of the mucosa, thrice daily for three weeks. The combined therapy is more effective than triamcinolone acetonide alone in Orabase®. By this method, clearance can be maintained by less frequent application or lower concentrations of the composition.

### SPONSORSHIP STATEMENT

The present invention is sponsored by Tehran University of Medical Sciences.

### BACKGROUND

1. Technical field

The present invention relates to a pharmaceutical formula prepared from a combination of triamcinolone acetonide and retinoic acid in Orabase® that can be used for the treatment of oral lichen planus, which manifests itself as keratotic, atrophic and erosive types that appears in oral mucosa.

2. Description of the Related Art

Oral lichen planus (OLP) is a chronic inflammatory disease with a 0.5-2.6% prevalence rate in the general population. It has a characteristically bilateral distribution and typically involves the buccal mucosa, gingiva, lip and tongue, that lesions of the reticular (Wickham's striae), papular, and plaque like forms are asymptomatic, although patients may complain of a slight roughness of the affected sites. While, atrophic and erosive lesions may produce various symptoms ranging from a mild burning sensation to intense pain that may interfere with speaking and cause difficulties during eating and swallowing [1, 2].

There is no known cure for this disease, because the etiology of it is unknown. It seems that an immunologically mediated process has a primary role in the development of this disease. Histologically, this is supported by a subepithelial band-formed infiltration dominated by T lymphocytes and macrophages and the degeneration of basal cells known as liquefaction degeneration. These data suggest OLP is a T-cell-mediated autoimmune disease in which auto cytotoxic CD8+ T cells trigger the apoptosis of oral epithelial cells [3, 4]. The other factors have been considered including: genetic predisposition, stress, hepatitis C [5]. Therefore its treatment is usually nonspecific and goals to alleviate symptoms and healing of severe lesions and other discomfort.

OLP is a chronic condition that can be difficult to manage and may be resistant to topical or systemic therapies. However, there are some topical and systemic medication such as steroids, retinoids, cyclosporine, tacrolimus, antimicrobials, immunomodulators, phenytoin and ultraviolet phototherapy for the treatment of OLP. They are all nonspecific and only acts temporarily in resolving the lesions [6]. Most of them reduce the lymphocytic exudate and formation of soluble inflammatory mediators, helping to maintain cellular membrane integrity, inhibit phagocytosis and release of lysozymes from granulocytes and stabilize the membranes of lysosomes that contain hydrolytic enzymes [5, 7]. In fact, the rationale behind their usage is their ability to modulate inflammation and immune response.

Corticosteroids are the most widely used agent in the treatment of OLP because of their action m suppressing cell mediated immune activity and to reduce pain and inflammation. They can be used topically, intralesionally or systemically. Topical steroids including triamcinolone acetonide 0.1% (KENALOG®), betamethasone valerate, fluocinonide (LIDEX®), clobestasol proprionate (TEMOV ATE®), and dexamethasone (DECADRON®) have provided the basis for the management of lichen planus [8,9,10,11].

In general, the greatest problem of using them in the oral cavity is to make them adhere to the mucosa for a sufficient period of time. There are forms as mouthwash, ointment, gel, or Orabase® that among them, Orabase® is more effective than gel or ointment. Orabase® is an adhesive paste containing gelatin, pectin, carboxy methylcellulose sodium and plasticized hydrocarbon gel such as polyethylene and mineral oil gel base. Many topical corticosteroids oral past formulation are available in market for management of different oral disorders. Accordingly, topical corticosteroids in adhesive pastes are the safest and most effective treatment of OLP for long-term use without systemic side effects [5]. Triamcinolone acetonide 0.1% (mid-potency steroid) is commonly used as an effective treatment of localized and symptomatic oral lichen planus in both forms of a mouth rinse and an orabase paste [5, 12].

Sahebjamee reported the effect of triamcinolone acetonide on decreasing keratotic areas due to its immunomodulatory effects [12]. A study on intralesional triamcinolone acetonide injection and its efficacy in treating ulcerative OLP reported that 84.4% of patients demonstrated complete response in ulceration size [10].

High-potency steroid mouthwashes such as disodium betamethasone phosphate or clobetasol propionate can be used in extensive OLP but these may be systemically absorbed leading to pituitary-adrenal axis suppression. Long-term use of topical steroids (more than 2 weeks continuously) may result in oral candidiasis, mucosal atrophy, and increase the systemic absorption. Antifungal therapy along with the topical steroids may be necessary or prophylactic antifungal therapy should be begun in patients with a history of fungal infection, immunosuppressed patients and those with prior steroid administration [5].

Retinoids are synthetic versions of vitamin A. Systemic, and topical retinoids have been used effectively in the treatment of OLP. They have anti-inflammatory, anti-keratinizing and immunomodulating effects. The anti-inflammatory properties refer to the ability of retinoids to inhibit migration of neutrophils from the blood vessels into the tissue (chemotaxis). Additionally, retinoids influence immune processes. Moreover, retinoids also inhibit migration of macrophages into OLP lesions. Therefore, its synthetic and natural analogues may be useful in the treatment of OLP and accelerating the healing process. Some studies reported that topical retinoid might be a suitable therapeutic agent in the treatment of hyperkeratotic OLP, leukoplakia [13].

Topical retinoid has been successfully used to treat OLP in some cases where corticosteroids have failed to achieve satisfactory results.

The efficacy of Retinoic acid 0.05% have been compared with triamcinolone acetonide 0.1%, both in Orabase®. The results showed that in nonkeratotic and even keratotic OLP, topical triamcinolone acetonide 0.1% reduced the severity of lesions more effectively than topical retinoic acid 0.05% [12]. Furthermore, the efficacy of retinoic acid in Orabase® (0.05%) has been compared with fluocinolone acetonide in Orabase® (0.1%), on atrophic and erosive OLP. The results showed that fluocinolone acetonide 0.1% reduced the severity of OLP better than retinoic acid 0.05% [10].

On the other hand, the primary use of retinoids is dissuaded and limited, due to their side effects and low remission rates. Both systemic and topical retinoids are suggested to be used as an adjuvant [2].

The corticosteroid-retinoid combination of the invention decrease inflammation by two entirely different mechanisms, acting in concert. One mechanism by which the corticosteroid works is by inhibiting the release of enzymes that initiate the inflammatory cascade, whereas the effect of the retinoid is less specific and perhaps interferes with the arachidic acid cascade. Among this is the ability of retinoid to promote wound healing and to stimulate the formation of angiogenesis, thus increasing the local blood supply [8, 13].

It presently reported that patients with long-standing oral lichen planus may develop squamous cell carcinoma of the mouth at a rate of 0.07%-2% over a 5-year period (14), that these indicate the need for long-term, appropriate follow-up and monitoring [5,15].

As mentioned previously, oral lichen planus is a chronic condition that is often subjected to medical treatment for long periods.

Long-term use of immunosuppressive agents could be a trigger for malignant transformation. For example, cyclosporine and tacrolimus can promote cancer progression [16, 17], while, a recent study of oral LP patients treated mostly with topical or systemic steroids, retinoids, therapeutic modalities did not affect the risk of malignant transformation [10-18].

According to population studies, Vitamin A may play a role in prevention of oral cancer and causes regression of premalignant leukoplakia [18].

According to the study on present invention the combination of triamcinolone acetonide and retinoic acid in Orabase® was more effective than triamcinolone acetonide in Orabase® in the treatment of OLP. It may be the results of synergistic effect of them.

Overall, a minority of patients may have the disease that closely mimics lichen planus, and are described as ‘lichenoid lesions (Examples include lichenoid drug reactions: anti-hypertensive agents, sulfonylureas, anti-malarials, gold, penicillamine, allopurinol, nonsteroidal anti-inflammatory agents and also lichenoid reactions seen in close proximity to amalgam restorations), chronic graft versus host disease (GVHD) and leukoplakia that they can be controlled and cleared by the treatments of the present invention [6].

### SUMMARY OF THE INVENTION

The object of this invention is to provide a different method of treating patients suffering from inflammatory conditions of the oral cavity such as OLP by a composition of triamcinolone acetonide and retinoic acid in Orabase®, which can control and clear OLP lesions more effective than triamcinolone acetonide alone. These two components have entirely different modes of action and when combined in a single formulation, have synergistic effects, which lead to more rapid clearing and are notably effective in treatment of OLP. Typically, the use of combination of corticosteroid and retinoid in Orabase® (thrice daily applications for two to three weeks) was more effective in decreasing the size of keratotic, atrophic and erosive OLP lesions than triamcinolone acetonide alone. The invention will be better understood from the following example.

### DETAILED DESCRIPTION

The object of this invention is to provide a different method of treating patients suffering from inflammatory conditions of the oral cavity such as OLP by a composition of triamcinolone acetonide and retinoic acid in Orabase®, which can control and clear OLP lesions more effective than triamcinolone acetonide alone. These two components have entirely different modes of action and when combined in a single formulation, have synergistic effects, which lead to more rapid clearing and are notably effective in treatment of OLP. Typically, the use of combination of corticosteroid and retinoid in Orabase® (thrice daily applications for two to three weeks) was more effective in decreasing the size of keratotic, atrophic and erosive OLP lesions than triamcinolone acetonide alone. The invention will be better understood from the following example.

### EXAMPLE 1

In this example, a formulation has been evaluated: 0.1% triamcinolone acetonide combined with 0.05% Retinoic acid in orabase: The drugs used in this study were 1) triamcinolone acetonide 0.1% and 2) combination of triamcinolone acetonide 0.1% with retinoic acid 0.05% both in an orabase paste, prepared in Department of Medicinal Chemistry, Faculty of Pharmacy, Tehran University of Medical Sciences, Tehran, Iran.

The composition of oral paste was gelatin, pectin, carboxy methylcellulose sodium, plasticized hydrocarbon gel prepared from polyethylene and mineral oil gel base in different ratios. To achieve desire viscosity and maximum of adherence to oral lesions different ratio of above mentioned ingredients and active compound (retinoic acid and triamcinolone acetonide) were mixed and tested. The best formulation for oral paste containing gelatin (16.5%), pectin (16.5%), carboxy methylcellulose sodium (16.7%), plasticized hydrocarbon gel (50.3%), gives satisfactory results. Appropriate of oral paste and active materials were mixed and filled in 30 g capacity tubes in absent of air and then labeled and kept in room temperature.

The composition of oral paste was gelatin, pectin, carboxy methylcellulose sodium, plasticized hydrocarbon gel prepared from polyethylene and mineral oil gel base in different ratios. To achieve desire viscosity and maximum of adherence to oral lesions different ratio of above mentioned ingredients and active compound (retinoic acid and triamcinolone acetonide) were mixed and tested. The best formulation for oral paste containing gelatin (16.5%), pectin (16.5%), carboxy methylcellulose sodium (16.7%), plasticized hydrocarbon gel (50.3%), gives satisfactory results. Appropriate of oral paste and active materials were mixed and filled in 30 g capacity tubes in absent of air and then labeled and kept in room temperature.

This study was a randomized control double-blind clinical trial. Forty patients with keratotic and atrophic/erosive OLP, including 28 females and 12 males were selected from those referred to the Department of Oral Medicine, Faculty of Dentistry, Tehran University of Medical Sciences. Inclusion criteria consisted of clinical and histopathological diagnosis of OLP based on ‘a modified definition of the World Health Organization (WHO) (19). An age range of 21-62 years, their mean age was 41±2 years signing an informed consent approved by the Ethics Committee of Tehran University of Medical Sciences.

Patients receiving immunosuppressive or immunomodulatory treatments or local drugs were eliminated or asked to discontinue their treatment for a minimum of one month before entering the investigation. Participants demonstrating histological signs of dysplasia, lichenoid drug reactions, drug consumption in the past month, pregnancy or breast-feeding women, uncontrolled diabetes, immunodeficiency or HCV infection was excluded from the study sample. In this study, 40 patients were randomly divided into groups of 20 (experimental and control) according to Block Randomization (www.randornization.com).

Experimental group (15 women and 5 men) was treated with the combination of triamcinolone acetonide 0.1% and retinoic acid 05%. In Orabase® (TRO) and control group (13 women and 7 men) received triamcinolone acetonide 0.1% in Orabase® (TO). The lesions size (sign) scored from 0 (no lesion) to 5 (large lesion). Table 1 shows the score range from 0 to 5 according to the criteria set by Thongprasom et al (10). A scaled tongue Blade (a wooden tongue blade was divided into equal 5 cm sections) was used to assess the size of the lesions, and the visual analogue scale (VAS) was used for assessment of pain (symptom). Pain scores or symptom stages were used to rank the severity of the subjects ‘pain and discomfort which ranged from 0 (showing no pain) to 100 mm (demonstrating extreme pain) (Table 2).

They were instructed to apply the medication on lesions three times a day for four weeks. All participants in both groups were examined at baseline, after 1, 2, 3 and four weeks of treatment, in order to assess the effectiveness of treatment. During each appointment, oral examination along with photography was performed and the patients' response rates were determined using pain scores and overall clinical improvement determined by Lesions score or sign stage. The symptomatic response for each patient was calculated by subtracting the final pain score from the initial score. The clinical response was estimated through subtraction of the lesions score obtained in the first and last examination sessions. Positive and negative values were considered as improvement and worsening, respectively. All data were recorded in the questionnaires at the end of every week.

Analysis and comparison of pain scores, size of the lesions' Clinical and symptomatic response rates between the two groups were performed by Mann-Whitney U-test using SPSS 13.0 computer software. A value of p<0.013 was considered to be statistically significant.

Forty patients, 28 women and 12 men, with the mean age of 41±2 years (range 21-62 years). Before the start of treatment, their lesions had already lasted for two months to eight years, with a mean of 28 months. The buccal mucosa was the most common site for OLP, followed by the gingiva, tongue, labial mucosa (Table 3). The patients in both groups were similar in age, and location of lesions. Before starting therapy, there was no statistically significant difference in the scores of signs and symptoms between the two groups (Table 3).

The use of medications in the two groups led to a decrease in pain and burning sensation severity of OLP, 95% of patients in TRO group and 90% of TO group were asymptomatic (no pain) after three weeks. The difference between the two groups was not statistically significant (P=0.71) (Table 4).

After 3 weeks of treatment, all patients in TRO group (100%) and 60% of TO be improved to score 1 & 0. Decrease in the size of keratotic, atrophic and erosive OLP lesions were significantly different in the two groups (P<0.0001) (Table 4). Thus, after 2 to 3 weeks, the decrease in the size of keratotic and atrophic/erosive lesions was greater in the experimental group compared to the control group.

The results of two-month follow up on the patients show that 10% of TRO group and 15% of TO group relapsed. Statistical analysis revealed that there was no significant difference between two groups (P=1) (Table 5). There were no side-effects in either of the groups throughout the entire study period. According to the discovery of the present invention, the retinoid enhances the efficacy of the corticosteroid in suppressing inflammation, which is characteristic of OLP lesions.

### EXAMPLE 2

Twenty patients with symptomatic geography tongue responded within two weeks from twice daily applications of the same combination as in Example 1.

### EXAMPLE 3

Some numbers of patients with leukoplakia responded by twice daily applications of the combination of triamcinolone 0.05% and retinoid acid 0.05% m an Orabase® for three to four weeks.

### EXAMPLE 4

A composition of 0.1% triamcinolone acetonide with 0.05% retinoid acid and nystatin 100000, units per gram can be used twice daily for immune suppressed patient with oral lichen planus.

Various oral inflammatory disorders included lichnoid reaction; chronic discoid lupus erythematosus (DLE) and chronic graft versus host disease (GVHD) may be controlled and cleared by the treatments of the present invention.

### Acknowledgment

This study was supported by the Dental Research center of Tehran University of Medical Sciences.

TABLE 1
Sign stage by Thongprasom
Stage Signs

| | |
|---|---|
| Score 0 | No lesion, normal mucosa |
| Score 1 | Mild white striae, no erythematous area |
| Score 2 | White striae with atrophic area less than 1 cm2 |
| Score 3 | White striae with atrophic area more than 1 cm2 |
| Score 4 | White striae with erosive area less than 1 cm2 |
| Score 5 | White striae with erosive area more than 1 cm2 |

TABLE 2
Symptoms stage or pain scores
Stage Symptoms

0 = Asymptomatic (no pain)
25 = Low level of symptoms, does not interfere with usual daily
activity (mild)
50 = Symptoms interfere with regular daily activity (moderate)
75 = Sore and painful; greatly interferes with regular daily activity
(severe)
100 = Impossible to live with the severe symptoms (very severe)

TABLE 3
Basic characteristics

| TRO group | TO group | |
|---|---|---|
| | | Sex |
| 5(25%) | 7(35%) | male |
| 15(75%) | 13(65%) | female |
| 45.8 ± 2 | 44.6 ± 2 | age |
| | | Location |
| 20(100%) | 20(100%) | Cheek |
| 12(60%) | 9(45%) | Gingiva |
| 5(25%) | 7(35%) | Tongue |
| 3(15%) | 2(10%) | lip |
| | | Pain score |
| | | Before intervention |
| 3(15%) | 3(15%) | Mild |
| 10(50%) | 9(45%) | Moderate |
| 4(20%) | 3(15%) | Severe |
| 3(15%) | 5(25%) | Very severe |
| | | Sign Score |
| | | Before intervention |
| — | — | Score1 |
| 0(0%) | 1(5%) | Score2 |
| 5(25%) | 2(10%) | Score3 |
| 8(40%) | 9(45%) | Score4 |
| 7(35%) | 8(40%) | Score5 |

TABLE 4
Results of treatment during 4 weeks

| 4 week | | 3 week | | 2 week | | 1 week | | |
|---|---|---|---|---|---|---|---|---|
| TRO | TO | TRO | TO | TRO | TO | TRO | TO | |
| | | | | | | | | Pain score |
| 20(100%) | 19(95%) | 19(95%) | 18(90%) | 12(60%) | 11(55%) | 12(60%) | 6(30%) | No pain |
| 0(0%) | 1(5%) | 1(5%) | 2(10%) | 5(25%) | 5(25%) | 7(35%) | 7(35%) | Mild |
| 0(0%) | 0(0%) | 0(0%) | 2(10%) | 3(15%) | 4(20%) | 1(5%) | 4(20%) | Moderate |
| 0(0%) | 0(0%) | 0(0%) | 1(5%) | 0(0%) | 0(0%) | 0(0%) | 2(10%) | severe |
| 0(0%) | 0(0%) | 0(0%) | 0(0%) | 0(0%) | 0(0%) | 0(0%) | 1(5%) | Very severe |
| 0.799 | | 0.718 | | 0.738 | | 0.024 | | P value |
| | | | | | | | | Sign score |
| 17(85%) | 6(30%) | 14(70%) | 4(20%) | 4(20%) | 0(0%) | 0(0%) | 0(0%) | Score 0 |
| 3(15%) | 11(55%) | 6(30%) | 8(40%) | 12(60%) | 5(25%) | 9(45%) | 1(5%) | Score 1 |
| 0(0%) | 3(15%) | 0(0%) | 6(30%) | 4(20%) | 7(35%) | 8(40%) | 3(15%) | Score 2 |
| 0(0%) | 0(0%) | 0(0%) | 1(5%) | 0(0%) | 5(25%) | 2(10%) | 8(40%) | Score 3 |
| 0(0%) | 0(0%) | 0(0%) | 1(5%) | 0(0%) | 3(15%) | 1(5%) | 6(30%) | Score 4 |
| 0(0%) | 0(0%) | 0(0%) | 0(0%) | 0(0%) | 0(0%) | 0(0%) | 2(10%) | Score 5 |

| | | | | |
|---|---|---|---|---|
| <0.001 | <0.001 | <0.001 | <0.001 | P value |

TABLE 5
Relapse rate between two groups

| P value | TO | TRO | relapse |
|---|---|---|---|
| — | 0(0%) | 0(0%) | 1 mouth |
| =1 | 3(15%) | 2(10%) | 2 mouth |

What is claimed is:

1. A composition for reducing or eliminating intraoral inflammation of oral lichen planus when topically administered on an affected area of the mucosal lesions, the composition comprising:
a triamcinolone acetonide and a retinoic acid in an Orabase with synergistic effect which are effective to suppress inflammation and control and clear said oral lichen planus lesions, wherein the composition is an oral paste, and wherein the Orabase® comprises gelatin, a pectin, a carboxy methyl cellulose sodium, and a plasticized hydrocarbon gel in different ratios, wherein the Orabase® composition comprises the gelatin in a ratio of 16.5%, pectin in a ratio of 16.5%, the carboxy methylcellulose sodium in a ratio of 16.7%, the plasticized hydrocarbon gel in a ratio of 50.3% and wherein the plasticized hydrocarbon gel is prepared from a polyethylene and a mineral oil gel, and wherein the composition of triamcinolone acetonide is between 0.01% to 5%, wherein the composition of retinoic acid is between 0.001% to 1%, and wherein the composition comprises traiamcinolone acetonide 0.1% and retinoic acid 0.05%.

2. The composition according to claim 1, wherein the composition is administrated to said affected area at least twice daily for at least two weeks.

3. (canceled)

4. The composition according to claim 1, wherein the composition is oral adhesive paste.

5. (canceled)

6. (canceled)

7. (canceled)

8. (canceled)
